(19) United States
(12) Patent Application Publication
(10) Pub. No.: US 2026/0035676 A1
(43) Pub. Date: Feb. 5, 2026

(54) TAQ DNA POLYMERASE MUTANT

(71) Applicants: FAPON LIFE SCIENCES INC., Lexington, MA (US); GUANGDONG FAPON BIOLOGICAL CO., LTD., Guangdong (CN)

(72) Inventors: Ning WU, Lexington, MA (US); Shengxi GUAN, Lexington, MA (US)

(21) Appl. No.: 19/287,814

(22) Filed: Aug. 1, 2025

Related U.S. Application Data
(60) Provisional application No. 63/678,532, filed on Aug. 1, 2024.

(51) Int. Cl. C12N 9/12; C12Q 1/686

(52) U.S. Cl. CPC C12N 9/1252; C12Q 1/686; C12Y 207/07007

(57) ABSTRACT

The present disclosure provides a mutant Taq DNA polymerase and the use thereof. In one embodiment, the mutant Taq DNA polymerase exhibits a DNA polymerase activity in a PCR reaction containing whole blood, high concentration salt or humic acid.

### CROSS-REFERENCE TO RELATED APPLICATIONS

This application claims priority to U.S. provisional application 63/678,532, filed Aug. 1, 2024, the disclosure of which is incorporated herein by reference.

### SEQUENCE LISTING

The sequence listing that is contained in the file named “088177-8006US02_seql.xml”, which is 2,730 bytes (as measured in Microsoft Windows) and was created on Jul. 30, 2025, is filed herewith by electronic submission and is incorporated by reference herein.

### FIELD OF THE INVENTION

The present disclosure relates generally to the field of molecular biology. In particular, the disclosure relates to a Taq DNA polymerase mutant.

### BACKGROUND

The polymerase chain reaction (PCR) is a method widely used for greatly amplifying the quantity of a very small sample of DNA. PCR is fundamental to many of the procedures used in genetic testing and research, making it one of the essential techniques in molecular biology. The majority of PCR methods rely on thermal cycling, which exposes reagents to repeated cycles of heating and cooling to permit different temperature-dependent reactions, specifically, DNA melting and enzyme-driven DNA replication. PCR employs two main reagents: primers, oligonucleotides that are complementary to the target DNA region, and a DNA polymerase. In the first step of PCR, the two strands of the DNA double helix are physically separated at a high temperature in the process of nucleic acid denaturation. In the second step, the temperature is lowered and the primers bind to the complementary region of DNA. The two DNA strands then become templates for DNA polymerase to enzymatically assemble a new DNA strand from free nucleotides.

Taq DNA polymerase, named after the thermophilic eubacterial microorganism Thermus aquaticus, from which it was originally isolated, is the first and most widely used thermostable DNA polymerase in PCR. Despite its extensive application in PCR reaction, Taq DNA polymerase still has some limitations. For example, Taq DNA polymerase has a lower specificity than other thermostable DNA polymerases and could add mismatches nucleotides to the sequence. Taq polymerase does not have 3′ to 5′ exonuclease proofreading activity, therefore mismatches nucleotides could not be corrected. Inhibitors in blood samples can prevent DNA binding, or directly inhibit Taq polymerase's activity. High concentration of salts can shield the negative charge of DNA molecules and therefore prevent their binding to polymerase molecules. As a result, Taq DNA polymerase may not work properly with certain samples, such as those containing whole blood or high concentration salt.

Protein engineering techniques, using site-specific or random mutagenesis, are powerful ways to create mutant enzymes from the known DNA polymerases. Several useful enzymes were successfully produced by these procedures. For example, a cold-sensitive mutant of Taq polymerase was developed with markedly reduced activity at 37° C., as compared with the wild type (WT) enzyme (Kermekchiev et al., 2003). This mutant may be applicable to hot start PCR. Another example is a mutant Taq polymerase with enhanced resistance to various inhibitors of PCR reactions, including whole blood, plasma, hemoglobin, lactoferrin, serum IgG, soil extracts, and humic acid (Kermekchiev et al., 2009). Mutational studies in the O-helix of Taq polymerase produced enzymes with reduced fidelity (Suzuki et al., 1997, 2000; Tosaka et al., 2001), which may be useful for error-prone PCR. There are continuing needs for the creation of novel mutant Taq polymerases with different substrate specificities, stabilities, and activities.

### SUMMARY

The present disclosure provides mutant Taq DNA polymerases that comprise a substitution of an amino acid residue corresponding to an alanine residue at position 516 of a wild-type Taq DNA polymerase, methods for synthesizing DNA using such mutant Taq DNA polymerases, and kits for use in such methods. The mutant Taq DNA polymerases, methods and kits disclosed herein address these and other needs.

In one aspect, the present disclosure provides a mutant Taq DNA polymerase comprising at least a substitution of an amino acid residue corresponding to the following residue of a wild-type Taq DNA polymerase comprising an amino acid sequence corresponding to SEQ ID NO: 1: a serine residue at position 515, an alanine residue at position 516, a serine residue at position 739, an alanine residue at position 743, or a methionine residue at position 747, wherein the amino acid residue is substituted with an arginine residue, a lysine residue, or a histidine residue.

In some embodiments, the mutant Taq DNA polymerase exhibits a DNA polymerase activity. In some embodiments, the mutant Taq DNA polymerase perform better than wild-type Taq DNA polymerase in DNA polymerase activity. In some embodiments, the mutant Taq DNA polymerase exhibits a DNA polymerase activity in a PCR reaction containing whole blood, plasma, serum, salt or humic acid. In some embodiments, the mutant Taq DNA polymerase exhibits a DNA polymerase activity in a PCR reaction containing (1) at least 5% whole blood, plasma or serum; (2) at least 60 mM salt; or (3) at least 5ng humic acid. In some embodiments, the mutant Taq DNA polymerase exhibits a DNA polymerase activity in a PCR reaction containing (1) any one content in 5%-20% whole blood, plasma or serum; (2) any one content in 60-300 mM salt; or (3) any one content in 5-25 ng humic acid. In some embodiments, the mutant Taq DNA polymerase exhibits a DNA polymerase activity in a PCR reaction containing (1) 5%, 10%, or 20% whole blood, plasma or serum; (2) 100mM, 150 mM, 200 mM, 250 mM or 300 mM salt; or (3) 5 ng, 10 ng, 15 ng, 20 ng, or 25 ng humic acid. In some embodiments, the salt is sodium or potassium salt, preferably potassium chloride.

In some embodiments, the amino acid residue corresponding to the serine residue at position 515 of the wild-type Taq DNA polymerase is substituted with an arginine residue or a lysine residue. In some embodiments, the amino acid residue corresponding to the alanine residue at position 516 of the wild-type Taq DNA polymerase is substituted with an arginine residue or a lysine residue. In some embodiments, the amino acid residue corresponding to the serine residue at position 739 of the wild-type Taq DNA polymerase is substituted with a lysine residue. In some embodiments, the amino acid residue corresponding to the alanine residue at position 743 of the wild-type Taq DNA polymerase is substituted with a lysine residue. In some embodiments, the amino acid residue corresponding to the methionine residue at position 747 of the wild-type Taq DNA polymerase is substituted with a lysine residue.

In some embodiments, the mutant Taq DNA polymerase further comprising another one or more substitutions of an amino acid residue. In some embodiments, the glutamate residue at position 742 of the wild-type Taq DNA polymerase is substituted with a lysine residue.

In some embodiments, the amino acid residue corresponding to the serine residue at position 515 of the wild-type Taq DNA polymerase is substituted with a lysine residue and the amino acid residue corresponding to the alanine residue at position 516 of the wild-type Taq DNA polymerase is substituted with a lysine residue. In some embodiments, the amino acid residue corresponding to the serine residue at position 515 of the wild-type Taq DNA polymerase is substituted with an arginine residue, and the amino acid residue corresponding to the alanine residue at position 516 of the wild-type Taq DNA polymerase is substituted with a lysine residue. In some embodiments, the amino acid residue corresponding to the alanine residue at position 516 of the wild-type Taq DNA polymerase is substituted with a lysine residue, and the amino acid residue corresponding to the serine residue at position 739 of the wild-type Taq DNA polymerase is substituted with a lysine residue. In some embodiments, the amino acid residue corresponding to the serine residue at position 515 of the wild-type Taq DNA polymerase is substituted with a lysine residue, and the amino acid residue corresponding to the alanine residue at position 516 of the wild-type Taq DNA polymerase is substituted with an arginine residue. In some embodiments, the amino acid residue corresponding to the serine residue at position 515 of the wild-type Taq DNA polymerase is substituted with an arginine residue, and the amino acid residue corresponding to the alanine residue at position 516 of the wild-type Taq DNA polymerase is substituted with an arginine residue. In some embodiments, the amino acid residue corresponding to the alanine residue at position 516 of the wild-type Taq DNA polymerase is substituted with an arginine residue, and the amino acid residue corresponding to the glutamate residue at position 742 of the wild-type Taq DNA polymerase is substituted with a lysine residue. In some embodiments, the amino acid residue corresponding to the alanine residue at position 516 of the wild-type Taq DNA polymerase is substituted with an arginine residue, and the amino acid residue corresponding to the methionine residue at position 747 of the wild-type Taq DNA polymerase is substituted with a lysine residue.

In some embodiments, the mutant Taq DNA polymerase disclosed herein has an amino acid sequence of at least 80 percent identity to SEQ ID NO: 1.

In some embodiments, the mutant Taq DNA polymerase disclosed herein further comprises one or more tags or linkers. In some embodiments, the mutant Taq DNA polymerase further comprises a tag at the N-terminus or C-terminus. In some embodiments, the mutant Taq DNA polymerase further comprises a His tag at the N-terminus or C-terminus. In some embodiments, the mutant Taq DNA polymerase further comprises GS as a linker.

In another aspect, the present disclosure provides a polynucleotide encoding the mutant Taq DNA polymerase disclosed herein.

In another aspect, the present disclosure provides a vector comprising the polynucleotide encoding the mutant Taq DNA polymerase disclosed herein.

In another aspect, the present disclosure provides a recombinant host cell suitable for producing the mutant Taq DNA polymerase disclosed herein. In some embodiments, the recombinant host cell comprises the polynucleotide encoding the mutant Taq DNA polymerase disclosed herein.

In another aspect, the present disclosure provides a method of producing a mutant Taq DNA polymerase. In some embodiments, the method comprises the steps of culturing the recombinant host cell disclosed herein, thereby giving a culture, and collecting the mutant Taq DNA polymerase from the culture obtained in the above step.

In another aspect, the present disclosure provides a kit for performing a polymerase chain reaction. In some embodiments, the kit comprises the mutant Taq DNA polymerase disclosed herein and a reaction buffer solution. In some embodiments, the kit further comprises a primer.

In another aspect, the present disclosure provides a method of performing a polymerase chain reaction. In some embodiments, the method comprises: incubating the mutant Taq DNA polymerase disclosed herein with a DNA template and a primer under a condition suitable for the mutant Taq DNA polymerase to perform the polymerase chain reaction, thereby synthesizing a DNA strand complementary to the DNA template.

### BRIEF DESCRIPTION OF DRAWING

The following drawings form part of the present specification and are included to further demonstrate certain aspects of the present disclosure. The disclosure may be better understood by reference to one or more of these drawings in combination with the detailed description of specific embodiments presented herein.

FIG. 1 illustrates the results of salt-resistance (100 mM KCl) qPCR test of wild-type Taq, mutant Taq with A516K substitution (EAA186) and mutant Taq with A516R substitution (EAA187).

FIG. 2 illustrates the results of salt-resistance (150 mM KCl) qPCR test of wild-type Taq, mutant Taq with A516K substitution (EAA186) and mutant Taq with A516R substitution (EAA187).

FIG. 3 illustrates the results of whole blood PCR test of wild-type Taq, mutant Taq with A516K substitution (EAA186) and mutant Taq with A516R substitution (EAA187).

FIG. 4 illustrates the results of humic acid PCR test of wild-type Taq, mutant Taq with A516K substitution (EAA186) and mutant Taq with A516R substitution (EAA187).

FIG. 5 illustrates the results of salt-resistance (150 mM, and 200 mM KCl) qPCR test of mutant Taq with S515K and A516K substitutions (EAA245), mutant Taq with S515R and A516K substitutions (EAA247), mutant Taq with A516K and S739K substitutions (EAA256), and mutant Taq with A516K substitution (EAA186).

FIG. 6 illustrates the results of whole blood PCR test of mutant Taq with A516K substitution (EAA186), mutant Taq with S515K and A516K substitutions (EAA245), and mutant Taq with S515R and A516K substitutions (EAA247).

FIG. 7 illustrates the results of humic acid PCR test of mutant Taq with A516K substitution (EAA186), mutant Taq with S515K and A516K substitutions (EAA245), and mutant Taq with S515R and A516K substitutions (EAA247).

FIG. 8 illustrates the results of salt-resistance (150 mM, 200 mM, and 250 mM KCl) qPCR test of mutant Taq with S515K and A516R substitutions (EAA246), mutant Taq with S515R and A516R substitutions (EAA248), mutant Taq with A516R and E742K substitutions (EAA258), mutant Taq with A516R and M747K substitutions (EAA260), and mutant Taq with A516R substitution (EAA187).

FIG. 9 illustrates the results of whole blood PCR test of mutant Taq with A516R substitution (EAA187), mutant Taq with S515K and A516R substitutions (EAA246), mutant Taq with S515R and A516R substitutions (EAA248), and mutant Taq with A516R and E742K substitutions (EAA258).

FIG. 10 illustrates the results of humic acid PCR test of mutant Taq with A516R substitution (EAA187), mutant Taq with S515K and A516R substitutions (EAA246), mutant Taq with S515R and A516R substitutions (EAA248), and mutant Taq with A516R and E742K substitutions (EAA258).

FIG. 11 illustrates the results of salt-resistance (100 mM KCl) qPCR test of wild-type Taq, mutant Taq with S515K substitution (EAA184) and mutant Taq with S515R substitution (EAA185).

FIG. 12 illustrates the results of salt-resistance (150 mM KCl) qPCR test of wild-type Taq, mutant Taq with S515R substitution (EAA185).

FIG. 13 illustrates the results of salt-resistance (100 mM KCl) qPCR test of wild-type Taq, mutant Taq with S739K substitution (EAA191).

FIG. 14 illustrates the results of salt-resistance (150 mM KCl) qPCR test of wild-type Taq, mutant Taq with M747K substitution (EAA194).

FIG. 15 illustrates the results of whole blood PCR tests of wild-type Taq, mutant Taq with S739K substitution (EAA191) and mutant Taq with M747K substitution (EAA194).

FIG. 16 illustrates the results of whole blood PCR tests of mutant Taq having multiple mutations ETaq1 to ETaq6 at four whole blood concentration gradients of 2.5%, 5%, 10%, and 20%.

FIG. 17 illustrates the results of humic acid PCR tests of mutant Taq having multiple mutations ETaq1 to ETaq6 at four humic acid concentration gradients of 0, 10 ng, 25 ng, and 50 ng.

### DETAILED DESCRIPTION OF THE INVENTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

### I. Definition

It is to be understood that both the foregoing general description and the following detailed description are exemplary and explanatory only and are not restrictive of the invention as claimed. In this application, the use of the singular includes the plural unless specifically stated otherwise. In this disclosure, the term “or” is used to mean “and/or” unless explicitly indicated to refer to alternatives only or the alternatives are mutually exclusive. As used herein “another” may mean at least a second or more. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise. Also, the use of the term “portion” can include part of a moiety or the entire moiety.

As used herein, the singular forms “a”, “an” and “the” include plural references unless the context clearly dictates otherwise.

The term “amino acid” as used herein refers to an organic compound containing amine (—NH₂) and carboxyl (—COOH) functional groups, along with a side chain specific to each amino acid. The names of amino acids are also represented as standard single letter or three-letter codes in the present disclosure.

As used herein, the term “amplifying” refers to a process whereby a portion of a nucleic acid is replicated using, for example, any of a broad range of primer extension reactions. Exemplary primer extension reactions include, but are not limited to, PCR. Unless specifically stated, “amplifying” refers to a single replication or to an arithmetic, logarithmic, or exponential amplification.

As used herein, “DNA polymerase” refers to a polypeptide that catalyzes the synthesis of DNA using an existing polynucleotide as a template.

The term “host cell” means a cell that has been transformed, or is capable of being transformed, with a nucleic acid sequence and thereby expresses a gene of interest. The term includes the progeny of the parent cell, whether or not the progeny is identical in morphology or in genetic make-up to the original parent cell, so long as the gene of interest is present.

As used herein, an “isolated” biological component (such as a nucleic acid, peptide or cell) has been substantially separated, produced apart from, or purified away from other biological components or cells of the organism in which the component naturally occurs, i.e., other chromosomal and extrachromosomal DNA and RNA, cells and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

The term “mutant” protein as used herein refers to a protein that has one or more amino acid substitutions, deletions (including truncations) or additions (including insertion) relative to a wild-type. A mutant protein may have less than 100% sequence identity to the amino acid sequence of a naturally occurring protein but may have any amino acid that is at least 80%, at least 85%, at least 90%, at least 95%, at least 97%, at least 98%, or at least 99% identical to the amino acid sequence of the naturally occurring protein.

The term “nucleic acid” or “polynucleotide” as used herein refers to deoxyribonucleic acids (DNA) or ribonucleic acids (RNA) and polymers thereof in either single-or double-stranded form. Unless otherwise indicated, a particular polynucleotide sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (see Batzer et al., Nucleic Acid Res. 19 (18): 5081 (1991); Ohtsuka et al., J. Biol. Chem. 260 (5): 2605-2608 (1985); and Rossolini et al., Mol. Cell. Probes 8 (2): 91-98 (1994)).

The term “operably linked” refers to an arrangement of elements wherein the components so described are configured so as to perform their usual function. Thus, a given signal peptide that is operably linked to a polypeptide directs the secretion of the polypeptide from a cell. In the case of a promoter, a promoter that is operably linked to a coding sequence will direct the expression of the coding sequence. The promoter or other control elements need not be contiguous with the coding sequence, so long as they function to direct the expression thereof. For example, intervening untranslated yet transcribed sequences can be present between the promoter sequence and the coding sequence and the promoter sequence can still be considered “operably linked” to the coding sequence.

“Percent (%) sequence identity” with respect to amino acid sequence (or nucleic acid sequence) is defined as the percentage of amino acid (or nucleic acid) residues in a candidate sequence that are identical to the amino acid (or nucleic acid) residues in a reference sequence, after aligning the sequences and, if necessary, introducing gaps, to achieve the maximum number of identical amino acids (or nucleic acids). Conservative substitution of the amino acid residues may or may not be considered as identical residues. Alignment for purposes of determining percent amino acid (or nucleic acid) sequence identity can be achieved, for example, using publicly available tools such as BLASTN, BLASTp (available on the website of U.S. National Center for Biotechnology Information (NCBI), see also, Altschul S. F. et al., J. Mol. Biol., 215 (3): 403-410 (1990); Stephen F. et al., Nucleic Acids Res., 25 (17): 3389-3402 (1997)), ClustalW2 (available on the website of European Bioinformatics Institute, see also, Higgins D.G. et al., Methods in Enzymology, 266:383-402(1996); Larkin M.A. et al., Bioinformatics (Oxford, England), 23 (21): 2947-8 (2007)), and ALIGN or Megalign (DNASTAR) software. A person skilled in the art may use the default parameters provided by the tool or may customize the parameters as appropriate for the alignment, such as for example, by selecting a suitable algorithm.

The term “polypeptide” or “protein” means a string of at least two amino acids linked to one another by peptide bonds. Polypeptides and proteins may include moieties in addition to amino acids (e.g., may be glycosylated) and/or may be otherwise processed or modified. Those of ordinary skill in the art will appreciate that a “polypeptide” or “protein” can be a complete polypeptide chain as produced by a cell (with or without a signal sequence) or can be a functional portion thereof. Those of ordinary skill will further appreciate that a polypeptide or protein can sometimes include more than one polypeptide chain, for example linked by one or more disulfide bonds or associated by other means. The term also includes amino acid polymers in which one or more amino acids are chemical analogs of a corresponding naturally occurring amino acid and polymers.

As used herein, a tag or fusion protein tag refers to a protein or peptide sequence genetically fused to a protein of interest. This tag aids in protein purification, detection, and sometimes enhances expression and solubility. It's a common technique in recombinant protein production, offering benefits like improved protein isolation, solubility, and refolding, as well as protection against proteolysis. Examples of fusion protein tag, without limitation, include His-tag, GST-tag, MBP-tag, GFP and Strep-tag.

As used herein, the term “primer” refers to an oligonucleotide, typically between about 10 to 100 nucleotides in length, capable of selectively binding to a specified target nucleic acid or “template” by hybridizing with the template. The primer can provide a point of initiation for template-directed synthesis of a polynucleotide complementary to the template, which can take place in the presence of appropriate enzyme(s), cofactors, substrates such as nucleotides and oligonucleotides and the like.

As used here, the term “primer extension reaction” refers to a reaction in which a polymerase catalyzes the template-directed synthesis of a nucleic acid from the 3′ end of a primer. The term “primer extension product” refers to the resultant nucleic acid. A non-limiting exemplary primer extension reaction is the polymerase chain reaction (PCR). The terms “extending” and “extension” refer to the template-directed synthesis of a nucleic acid from the 3′ end of a primer, which is catalyzed by a polymerase.

The term “recombinant” when used with reference to a polypeptide (e.g., antibody, antigen) or a polynucleotide, refers to a polypeptide or polynucleotide that is produced by a recombinant method. A “recombinant polypeptide” includes any polypeptide expressed from a recombinant polynucleotide. A “recombinant polynucleotide” includes any polynucleotide which has been modified by the introduction of at least one exogenous (i.e., foreign, and typically heterologous) nucleotide or the alteration of at least one native nucleotide component of the polynucleotide and need not include all of the coding sequence or the regulatory elements naturally associated with the coding sequence. A “recombinant vector” refers to a non-naturally occurring vector, including, e.g., a vector comprising a recombinant polynucleotide sequence.

As used herein, “substitution” refers to the replacement of at least one base, nucleobase, nucleoside, nucleotide or amino acid with a different base, nucleobase, nucleoside, nucleotide or amino acid.

As used herein, a “vector” refers to a nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

II. Mutant Taq DNA Polymerase and Production Thereof

DNA polymerases are polypeptides that catalyze the synthesis of DNA using an existing polynucleotide as a template. DNA polymerases include DNA-dependent polymerases, which use DNA as a template, or RNA-dependent polymerases, such as reverse transcriptase (RT), which use RNA as a template.

Based on sequence homology, bacterial DNA polymerases can be subdivided into seven different families: A, B, C, D, X, Y, and RT. DNA-dependent DNA polymerases fall into one of six families (A, B, C, D, X, and Y), with most falling into one of three families (A, B, and C). See, e.g., Ito et al. (1991) Nucleic Acids Res. 19:4045-4057; Braithwaite et al. (1993) Nucleic Acids Res. 21:787-802; Filee et al. (2002) J. Mol. Evol. 54:763-773; and Alba (2001) Genome Biol. 2:3002.1-3002.4. Certain DNA polymerases may be single-chain polypeptides (e.g., certain family A and B polymerases) or multi-subunit enzymes (e.g., certain family C polymerases) with one of the subunits having polymerase activity.

Family A polymerases (“Pol A”) include both replicative and repair polymerases. Replicative members from this family include T7 DNA polymerase and the eukaryotic mitochondrial DNA Polymerase y. Among the repair polymerases are E. coli DNA Pol I, Thermus aquaticus Pol I (Taq DNA polymerase), and Bacillus stearothermophilus Pol I. Excision repair and processing of Okazaki fragments generated during lagging strand synthesis are performed by the repair polymerases. Because most thermostable Pol A enzymes do not possess the 3′ to 5′ exonuclease activity, they are incapable of proofreading the newly synthesized nucleic acid strand and consequently have high error rates.

Family B polymerases (“Pol B”) are substantially replicative polymerases including the major eukaryotic DNA polymerases α, δ, ϵ, and also DNA polymerase ζ. Pol B polymerases also include DNA polymerases encoded by some bacteria and bacteriophages, of which the best characterized are from T4, PM29 and RB69 bacteriophages. Pol B enzymes are involved in both leading and lagging strand synthesis and are noteworthy for their remarkable accuracy during replication as many have strong 3′-5′ exonuclease activity the exceptions being DNA polymerase α and ζ which lack proofreading activity.

Family C polymerases are the major replicative polymerases in bacteria. DNA polymerase III is the main family C polymerase involved in E. coli DNA replication.

Family D polymerases are present in Euryarchaeota, a subdomain of archaea, and are mainly replicative. This family of polymerases is not clearly defined but studies of Pyrococcus furiosus DNA polymerase II suggest this enzyme is a replicative polymerase

Family X polymerases include the eukaryotic polymerase pol β, along with others such as pol μ, pol λ, pol σ and terminal deoxynucleotidyl transferase. Polymerase β performs short patch repair of damaged DNA by fixing alkylated, oxidized or abasic sites that have formed due to DNA damage. Pol λ and pol μ are involved in the rejoining of breaks that have occurred in double strands of DNA due to hydrogen peroxide (in the case of pol λ) and ionizing radiation (in the case of pol μ). Terminal deoxynucleotidyl transferase is only found in lymphoid tissue and adds non-templated nucleotides at V(D)J junctions, to provide diversity.

Family Y polymerases have a low fidelity for intact DNA strands and are capable of replicating damaged DNA. One example of a family Y polymerase is pol IV, an error-prone polymerase that has no 3′ to 5′ proofreading activity and is involved in mutagenesis. The enzyme is expressed by a gene (dinB) that is switched on when polymerases stall at the replication fork. This interferes with the processivity of pol III which acts as a checkpoint, stopping replication and allowing time for DNA to be repaired. Cells that lack dinB are at an increased risk of developing mutations caused by agents that damage DNA. Pol V also belongs to the Y family of polymerases and allows DNA damage to be bypassed in order for replication to continue.

DNA polymerases are roughly shaped like a hand with a thumb, palm and fingers. The thumb is involved in binding and moving double-stranded DNA. The palm carries the polymerase active site, whereas the fingers bind substrates (template DNA and nucleoside triphosphates). The exonuclease activity is in a separate protein domain. Mg²⁺ is a cofactor.

Thermostable DNA polymerases are DNA polymerases that originate from thermophiles, usually bacterial or archacal species, and are therefore thermostable. Among the bacterial thermostable DNA polymerases, Taq polymerase, Tfl polymerase, Tma polymerase, Tne polymerase, Tth and Bst polymerase are used in polymerase chain reaction and related methods for the amplification and modification of DNA.

Taq DNA polymerase is the first and most widely used thermostable DNA polymerase in PCR. Despite its extensive application, Taq DNA polymerase has some limitations, such as a lower specificity than other thermostable DNA polymerases, and no 3′ to 5′ exonuclease proofreading activity. In addition, Taq DNA polymerase requires cofactors, such as Mg²⁺ ion, to work properly. As a result, Taq DNA polymerase may not work properly with samples that contains PCR inhibitors, such as whole blood samples and samples of high salt concentration.

Mutant Taq DNA polymerases with improved functions have been generated by protein engineering techniques. For example, a cold-sensitive mutant of Taq polymerase, which may be applicable to hot start PCR, was developed with markedly reduced activity at 37° C., as compared with the wild type (WT) enzyme (Kermekchiev et al., 2003). Another example is a mutant Taq polymerase with enhanced resistance to various inhibitors of PCR reactions, including whole blood, plasma, hemoglobin, lactoferrin, serum IgG, soil extracts, and humic acid (Kermekchiev et al., 2009). Mutational studies in the O-helix of Taq polymerase produced enzymes with reduced fidelity (Suzuki et al., 1997, 2000; Tosaka et al., 2001), which may be useful for error-prone PCR.

### A. Mutant Taq DNA Polymerase

The present disclosure in one aspect provides a mutant Taq DNA polymerase that polymerase exhibits an improved DNA polymerase activity in the presence of various inhibitors of PCR reactions, including whole blood and humic acid. In some embodiments, the mutant Taq DNA polymerase comprises a substitution of an amino acid residue corresponding to an alanine residue at position 516 of a wild-type Taq DNA polymerase comprising an amino acid sequence corresponding to SEQ ID NO: 1. In some embodiments, the alanine residue at position 516 is substituted with a polar amino acid. In some embodiments, the substitution of the alanine residue at position 516 is selected from the group consisting of: an arginine residue, a lysine residue, an asparagine residue, a glutamine residue, a histidine residue, an aspartate residue and a glutamate residue. In some embodiments, the amino acid residue corresponding to an alanine residue at position 516 of a wild-type Taq DNA polymerase is substituted with an arginine residue or a lysine residue. In some embodiments, the amino acid residue corresponding to an alanine residue at position 516 of a wild-type Taq DNA polymerase is substituted with an asparagine residue, a glutamine residue, or a histidine residue. In some embodiments, the amino acid residue corresponding to an alanine residue at position 516 of a wild-type Taq DNA polymerase is substituted with an aspartate residue or a glutamate residue.

Protein sequence of wild type Taq DNA polymerase (SEQ ID NO: 1)

MRGMLPLFEP KGRVLLVDGH HLAYRTFHAL KGLTTSRGEP
VQAVYGFAKS LLKALKEDGD AVIVVEDAKA PSFRHEAYGG
YKAGRAPTPE DFPRQLALIK ELVDLLGLAR LEVPGYEADD
VLASLAKKAE KEGYEVRILT ADKDLYQLLS DRIHVLHPEG
YLITPAWLWE KYGLRPDQWA DYRALTGDES DNLPGVKGIG
EKTARKLLEE WGSLEALLKN LDRLKPAIRE KILAHMDDLK
LSWDLAKVRT DLPLEVDEAK RREPDRERLR AFLERLEFGS
LLHEFGLLES PKALEEAPWP PPEGAFVGFV LSRKEPMWAD
LLALAAARGG RVHRAPEPYK ALRDLKEARG LLAKDLSVLA
LREGLGLPPG DDPMLLAYLL DPSNTTPEGV ARRYGGEWTE
EAGERAALSE RLFANLWGRL EGEERLLWLY REVERPLSAV
LAHMEATGVR LDVAYLRALS LEVAEEIARL EAEVERLAGH
PENLNSRDQL ERVLEDELGL PAIGKTEKTG KRSTSAAVLE
ALREAHPIVE KILQYRELTK LKSTYIDPLP DLIHPRTGRL
HTRENQTATA TGRLSSSDPN LQNIPVRTPL GQRIRRAFIA
EEGWLLVALD YSQIELRVLA HLSGDENLIR VFQEGRDIHT
ETASWMFGVP REAVDPLMRR AAKTINFGVL YGMSAHRLSQ
ELAIPYEEAQ AFIERYFQSF PKVRAWIEKT LEEGRRRGYV
ETLEGRRRYV PDLEARVKSV REAAERMAEN MPVQGTAADL
MKLAMVKLEP RLEEMGARML LQVHDELVLE APKERAEAVA
RLAKEVMEGV YPLAVPLEVE VGIGEDWLSA KE

In some embodiments, the mutant Taq DNA polymerase disclosed herein further comprises a substitution of an amino acid residue corresponding to a serine residue at position 515 of a wild-type Taq DNA polymerase with an arginine residue or a lysine residue. In some embodiments, the mutant Taq DNA polymerase disclosed herein further comprises a substitution of an amino acid residue corresponding to a serine residue at position 739 of a wild-type Taq DNA polymerase with a lysine residue. In some embodiments, the mutant Taq DNA polymerase disclosed herein further comprises a substitution of an amino acid residue corresponding to a glutamate residue at position 742 of a wild-type Taq DNA polymerase with a lysine residue. In some embodiments, the mutant Taq DNA polymerase disclosed herein further comprises a substitution of an amino acid residue corresponding to a methionine residue at position 747 of a wild-type Taq DNA polymerase with a lysine residue.

In some embodiments, the mutant Taq DNA polymerase disclosed herein has an amino acid sequence of at least 70, 75, 80, 85, 90, 95, or 99 percent identity to SEQ ID NO: 1.

In some embodiments, the mutant Taq DNA polymerase displays an enhanced DNA polymerase activity of at least 10, 20, 30, 40, 50, 60, 70, 80, 90 or 100 percent as compared to the wild type Taq DNA polymerase in the presence of high salt concentration, whole blood or humic acid.

### B. Methods of Production

The mutant Taq DNA polymerase according to the present disclosure can be prepared recombinantly, by expression from e.g. a nucleic acid construct encoding for the mutant Taq DNA polymerase, for example as described in Molecular Cloning: A Laboratory Manual, 4th edition (Sambrook et al., 2001), the entire contents of both of which are hereby incorporated by reference.

In one embodiment, DNA encoding the wild type Taq DNA polymerase can be isolated using conventional procedures (e.g., by using oligonucleotide probes that are capable of binding specifically to the Taq DNA polymerase gene). The encoding DNA may also be obtained by synthetic methods. The encoding polynucleotide can then be mutated at the site of interest by site-directed mutagenesis at the selected codons encoding the alanine residue at position 516 or other residues of interest. The mutant encoding polynucleotide can be inserted into a vector to generate a polynucleotide encoding the mutant Taq DNA polymerase using recombinant techniques known in the art. Many vectors are available. The vector components generally include, but are not limited to, one or more of the following: a signal sequence, an origin of replication, one or more marker genes, an enhancer element, a promoter (e.g. T7, SV40, CMV, EF-1α), and a transcription termination sequence, which are operably linked to the mutant encoding polynucleotide.

Vectors comprising the polynucleotide sequence encoding the mutant Taq DNA polymerase can be introduced to a host cell for cloning or gene expression. Suitable host cells for cloning or expressing the DNA in the vectors herein are the prokaryote (e.g., E. coli), yeast (e.g., Saccharomyces cerevisiae), or higher eukaryote cells (e.g., mammalian host cell lines).

Host cells are transfected with the above-described expression or cloning vectors for mutant Taq DNA polymerase production and cultured in conventional nutrient media modified as appropriate for inducing promoters, selecting transformants, or amplifying the genes encoding the desired sequences.

In certain embodiments, the mutant Taq DNA polymerase of the present disclosure may be purified. The term “purified,” as used herein, is intended to refer to a composition, isolatable from other components, wherein the protein is purified to any degree relative to its naturally-obtainable state. A purified protein therefore also refers to a protein, free from the environment in which it may naturally occur. Where the term “substantially purified” is used, this designation will refer to a composition in which the protein or peptide forms the major component of the composition, such as constituting about 10%, about 20%, about 30%, about 40%, about 50%, about 60%, about 70%, about 80%, about 90%, about 95% or more of the proteins (e.g., by weight) in the composition.

Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. Other methods for protein purification include, precipitation with ammonium sulfate, PEG, or by heat denaturation, followed by centrifugation; gel filtration, reverse phase, hydroxylapatite and affinity chromatography; and combinations of such and other techniques.

### III. Compositions and Kits

Also provided in the present disclosure are compositions and kits comprising the mutant Taq DNA polymerase described herein. Such compositions and kits comprise, in addition to the mutant Taq DNA polymerase described herein, components usable for DNA synthesis, such as primer, deoxyribonucleotide, and reaction buffer.

In one embodiment, the composition or kit according to the present disclosure may include at least one primer, at least one deoxyribonucleotide, and/or a reaction buffer solution in addition to the mutant Taq DNA polymerase described herein.

The primer may be an oligonucleotide having a nucleotide sequence complementary to the template DNA, and is not particularly limited as long as it anneals to the template DNA under the reaction conditions used. The primer may be oligonucleotide having a random sequence (random primer).

The length of the primer is preferably at least six nucleotides since a specific annealing process is performed, and more preferably at least 10 nucleotides. The length of the primer is preferably at most 100 nucleotides and more preferably at most 30 nucleotides in terms of the synthesis of oligonucleotide. The oligonucleotide can be synthesized, for example, according to the phosphoramidite method by the DNA synthesizer 394 (manufactured by Applied Biosystems Inc). The oligonucleotide may be synthesized according to any other process, such as the triester phosphate method, H-phosphonate method, or thiophosphate method. The oligonucleotide may be oligonucleotide derived from a biological specimen, and for example, may be prepared such that it is isolated from restricted endonuclease digest of DNA prepared from a natural specimen.

As used herein, deoxyribonucleotide refers to phosphate groups bonded to deoxyribose bonded to organic bases by the phosphoester bond. A natural DNA includes four different nucleotides. The nucleotides respectively consisting of adenine, guanine, cytosine and thymine bases can be found in the natural DNA. The adenine, guanine, cytosine and thymine bases, are respectively abbreviated as A, G, C and T. The deoxyribonucleotide includes free monophosphate, diphosphate and triphosphate (more specifically, the phosphate groups each includes one, two or three phosphate portions). Therefore, the deoxyribonucleotide includes deoxyribonucleotide triphosphate (for example, dATP, dCTP, dITP, dGTP and dTTP) and derivatives thereof. The deoxyribonucleotide derivative includes [αS] dATP, 7-deaza-dGTP, 7-deaza-dATP and a deoxynucleotide derivative showing resistance against the decomposition of nucleic acid. The nucleotide derivative includes, for example, deoxyribonucleotide labeled in such a manner that can be detected by a radioactive isotope such as ³²P or ³⁵S, a fluorescent portion, a chemiluminescent portion, a bioluminescent portion or an enzyme.

Deoxyribonucleotide triphosphate, as used herein, refers to a nucleotide of which the sugar portion is composed of deoxyribose, and having a triphosphate group. A natural DNA includes four different nucleotides which respectively has adenine, guanine, cytosine and thymine as the base portion. The deoxyribonucleotide triphosphate contained in an exemplary composition or kit of the present disclosure is a mixture of four deoxyribonucleotides triphosphate, dATP, dCTP, dGTP, and dTTP.

As used herein, the reaction buffer solution means a solution suitable for the mutant Taq DNA polymerase disclosed herein to perform DNA synthesis. In one embodiment, the reaction buffer includes a buffer agent or a buffer agent mixture and may further include divalent cations and monovalent cations. In one embodiment, the reaction buffer contained in the composition or kit is a 5× or 10× buffer solution, i.e., the buffer solution needs to be diluted 5 or 10 times in a reaction for DNA synthesis. In one embodiment, the reaction buffer solution 1× contains 20 mM Tris 8.0, 6 mM (NH₄) SO₄, 2 mM MgCl₂, and 3% DMSO.

### IV. Method of Use

In another aspect, the present disclosure provides methods of using the mutant Taq DNA polymerase as disclosed herein for DNA synthesis.

In one embodiment, the method for synthesizing DNA using the composition disclosed herein, comprises the steps of:

A) preparing a solution comprising the mutant Taq DNA polymerase disclosed herein, at least one primer, at least one deoxyribonucleotide, and DNA serving as a template; and

B) incubating the solution prepared in the step A) under a condition suitable for the mutant Taq DNA polymerase to perform primer extension, i.e., synthesizing DNA using the DNA as the template.

In one method disclosed herein, the DNA synthesis reaction may include one kind of template or a plurality of different templates having different nucleotide sequences. When a specific primer for a particular template is used, primer extension products from the plurality of different templates in the nucleic acid mixture can be produced. The plurality of templates may be present in the different nucleic acids or the same nucleic acid. The DNA, which is a template to which the method disclosed herein is applicable, is not particularly limited. Examples of the DNA are an group of DNA molecules in all of DNAs in a specimen, a group of DNA molecules such as plasmid DNA or genomic DNA, or particular group of DNA molecules (for example, a group of DNA molecules having a common nucleotide sequence motif, a group of DNA molecules concentrated by means of the subtraction process).

In some embodiments, the DNA serving as the template may be included in a specimen derived from an organism such as cells, tissues or blood, or a specimen such as food, soil or waste water which possibly includes organisms. Further, the DNA may be included in a nucleic acid-containing preparation obtained by processing such a specimen or the like according to the conventional process. Examples of the preparation is homogenized cells, and a specimen obtained by fractioning the homogenized cells, all of DNAs in the specimen, or a group of particular DNA molecules, for example, a specimen in which genomic DNA is enriched, and the like.

The amount of the mutant Taq DNA polymerase to be used in the method disclosed herein is not particularly limited. In the case where the DNA synthesis reaction is performed with 20 μL of the reaction solution, the amount of the mutant Taq DNA polymerase can be 0.02-20 μg, or 1-10 μg, or 2-5 μg.

The concentration of the primer used in the method disclosed herein is not particularly limited. The concentration is preferably at least 0.1 μM, 0.2 μM or 0.3 μM in the DNA synthesis reaction.

The conditions which are suitable for the mutant Taq DNA polymerase to perform DNA synthesis reaction, i.e., satisfactory for synthesizing the primer extension strand complementary to the template DNA are not particularly limited. The first step in a typical PCR amplification includes heat denaturation of the double-stranded target nucleic acid. The exact conditions required for denaturation of the sample nucleic acid depends on the length and composition of the sample nucleic acid. Typically, an incubation at 90-100° C. for about 10 seconds up to about 4 minutes is effective to fully denature the sample nucleic acid. The initial denaturation step can serve as the pre-reaction incubation to reactivate the DNA polymerase. The annealing temperature used in PCR amplification is typically about 50-75° C. The primer extension temperature used in PCR amplification is typically above 50° C. or above 60° C.

The following examples are provided to better illustrate the claimed invention and are not to be interpreted in any way as limiting the scope of the invention. All specific compositions, materials, and methods described below, in whole or in part, fall within the scope of the invention. These specific compositions, materials, and methods are not intended to limit the invention, but merely to illustrate specific embodiments falling within the scope of the invention. One skilled in the art may develop equivalent compositions, materials, and methods without the exercise of inventive capacity and without departing from the scope of the invention. It will be understood that many variations can be made in the procedures herein described while still remaining within the bounds of the invention. It is the intention of the inventors that such variations are included within the scope of the invention.

### EXAMPLE 1

This example shows the activity of the mutant Taq DNA polymerase in salt resistance PCR reaction.

### Preparation of Cell Lysates

Taq genes with designed mutations were cloned into the pET-28a vector for expression. 25 μL of Nico21 (DE3) cells were transformed with purified plasmids encoding Taq variants. Then 50 μL of seed culture was inoculated into 3 mL of pre-warmed LB media, which was incubated overnight at 37° C. with shaking at 250 rpm. Inoculate 3 mL of fresh TB broth with 30 μL of overnight culture the second day morning, and let it grow until an OD₆₀₀ of ˜0.6 is reached. Induce the cultures to express protein by adding IPTG at a final concentration of 0.5 mM followed by overnight growth at 18° C.

Cells were harvested by centrifugation at 4000 rpm, 4° C. for 5 min. Wet cell pastes were resuspended with BugBuster HT lysis buffer following the manufacturer's instructions. The cell suspension was then incubated on a plate shaker at a slow setting (300 rpm) for 20 min at room temperature, at the end of which the extract should not be viscous. Then, a heat treatment was performed at 75 C for 15 min. Cell lysates were cleared by centrifugation at 4000 rpm for 30 min at 4 C.

qPCR Based Salt Resistant Assay

2 μL of cleared cell lysates were added into a 20 μL reaction, with 1× PCR buffer (20 mM Tris-Cl 8.0, 6 mM (NH₄)SO₄, 2 mM MgCl₂, and 3% DMSO), various concentrations of KCl (60, 100, 200, 250, and 300 mM final), 200 μM of dNTPs, 0.3 μM of forward and reverse primers, and 0.15 μM of probe. 2 pg of human Jurkat cDNA was added as the template. The reactions were mixed, spanned at 2500 rpm for 2 min at 4 C, and then ran on a Biorad CFX-96 real time PCR system. Data were analyzed with provided software package.

### Results

The resistance of various Taq mutants to KCl of different concentrations were tested. The results are shown in the Table below. The wild type Taq DNA polymerase control can tolerate 200 mM of KCl under the same condition (cell lysates). The relative salt resistance is calculated by the following formula: KCl resistance_Taq mut/KCl resistance_Taq wt. “Inactive” means the variant showed no activity in the presence of the lowest salt concentration tested (60 mM), so its relative salt resistance is lower than 60/200=0.3. Please note that the absolute concentration of salt that a Taq polymerase can tolerate may be different when it's measured in the format of cell lysates or purified protein. The relative salt resistance was used to compare variants' salt resistance capacity when they were tested under different conditions. The data shown in this table were all obtained with proteins in cell lysates.

| Taq Mutant | KCl resistance (mM) | Resistance relative to WT |
|---|---|---|
| S515K | 250 | 1.25 |
| S515R | 300 | 1.5 |
| A516K | 250 | 1.25 |
| A516R | 300 | 1.5 |
| N580K | Inactive | <0.3 |
| N580R | Inactive | <0.3 |
| N583K | Inactive | <0.3 |
| N583R | Inactive | <0.3 |
| V586K | 60 | 0.3 |
| V586R | 100 | 0.5 |
| H784K | Inactive | <0.3 |
| H784R | Inactive | <0.3 |
| S739K | 300 | 1.5 |
| A743K | 300 | 1.5 |
| M747K | 300 | 1.5 |

### EXAMPLE 2

This example shows the activity of purified mutant Taq DNA polymerase in salt resistance PCR reaction (100 mM or 150 mM KCl). The protocol of qPCR-based salt resistant assay is similar to the method described in EXAMPLE 1 except the cleared cell lysate was replaced by purified Taq.

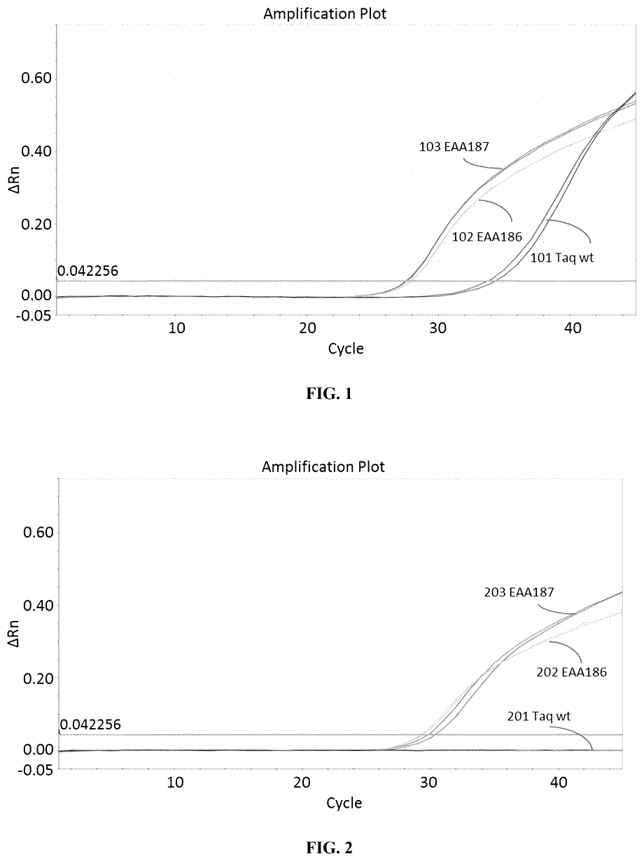

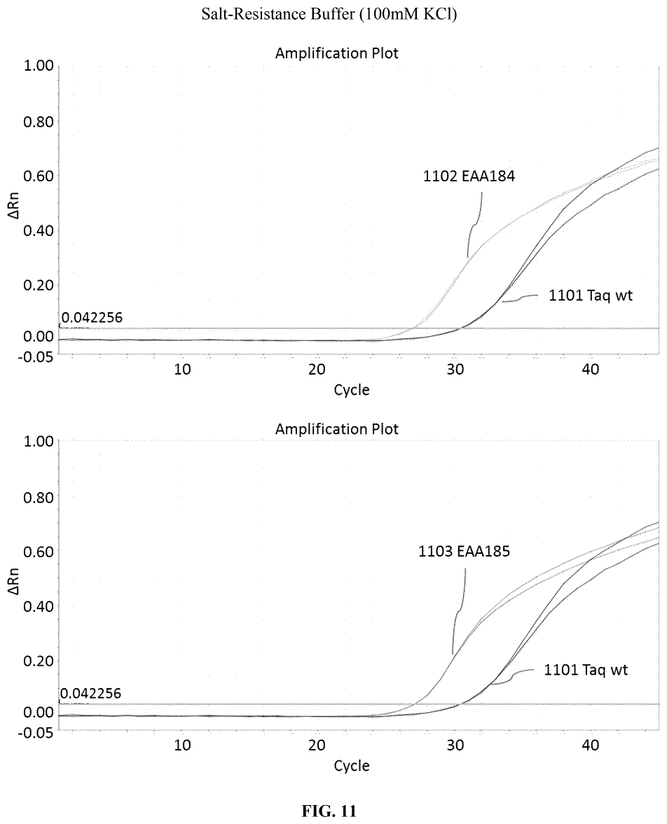

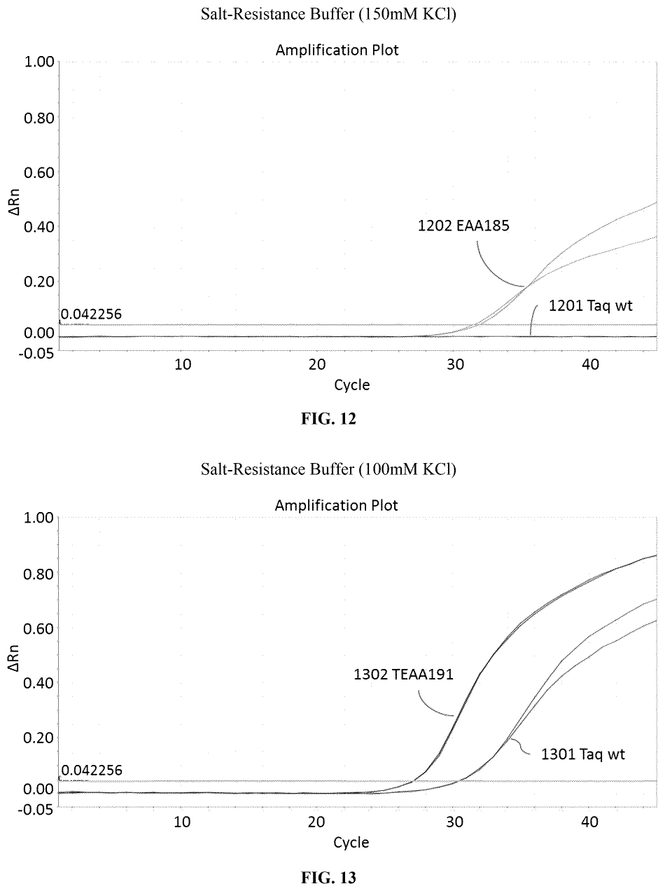

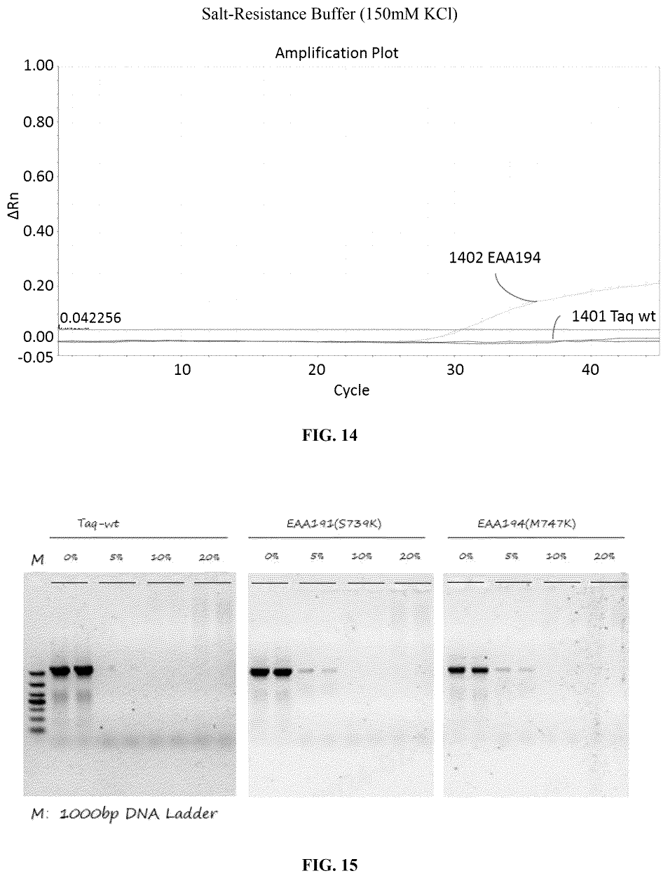

As shown in FIGS. 1, 2, 11-14, mutant Taq DNA polymerases with A516K substitution (EAA186), with A516R substitution (EAA187), with S515K substitution (EAA184), with S515R substitution (EAA185), with S739K substitution (EAA191), or with M747K substitution (EAA194), demonstrated enhanced salt resistance as compared to wild-type Taq DNA polymerase.

### EXAMPLE 3

This example illustrates the enhanced DNA synthesis activity of the mutant Taq DNA polymerase in whole blood PCR reaction.

PCR based whole blood assay

25 ng of purified Taq variants were added into a 25 uL reaction, with 1× PCR buffer (20 mM Tris-Cl 8.0, 6 mM (NH₄)SO₄, 2 mM MgCl₂, and 3% DMSO), various concentrations of human whole blood as inhibitors (0, 5%, 10% and 20% final), 200 uM of dNTPs, and 0.3 uM of forward and reverse primers. 0.5 pg of lambda DNA was added as the template to amplify a 1 kb PCR amplicon. The reactions were mixed, spanned at 2500 rpm for 2 min at 4 C, and then ran on a PCR system. The thermocycling parameters were as follows: 95° C. for 5 min; 95° C. for 30 seconds; 58° C. for 30 seconds; and 72° C. for 1 minute; for 30 cycles.

### Results

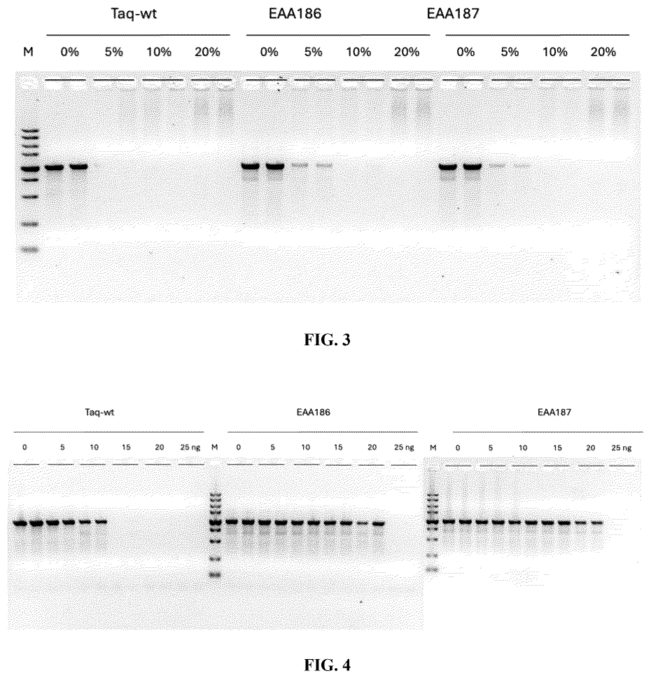

As shown in FIGS. 3 and 15, mutant Taq DNA polymerases with A516K substitution (EAA186), with A516R substitution (EAA187), with S739K substitution (EAA191), or with M747K substitution (EAA194) demonstrated enhanced DNA synthesis activity as compared to wild-type Taq DNA polymerase in whole blood PCR reaction.

### EXAMPLE 4

This example illustrates the enhanced DNA synthesis activity of the mutant Taq DNA polymerase in humic acid PCR reaction.

PCR based humic acid assay

25 ng of purified Taq variants were added into a 25 uL reaction, with 1× PCR buffer (20 mM Tris-Cl 8.0, 6 mM (NH₄)SO₄, 2 mM MgCl₂, and 3% DMSO), various concentrations of humic acids as inhibitors (0, 10, 25, and 50ng final), 200 uM of dNTPs, and 0.3 uM of forward and reverse primers. 0.5 pg of lambda DNA was added as the template to amplify a 1 kb PCR amplicon. The reactions were mixed, spanned at 2500 rpm for 2 min at 4C, and then ran on a PCR system. The thermocycling parameters were as follows: 95° C. for 5 min; 95° C. for 30 seconds; 58° C. for 30 seconds; and 72° C. for 1 minute; for 30 cycles.

### Results

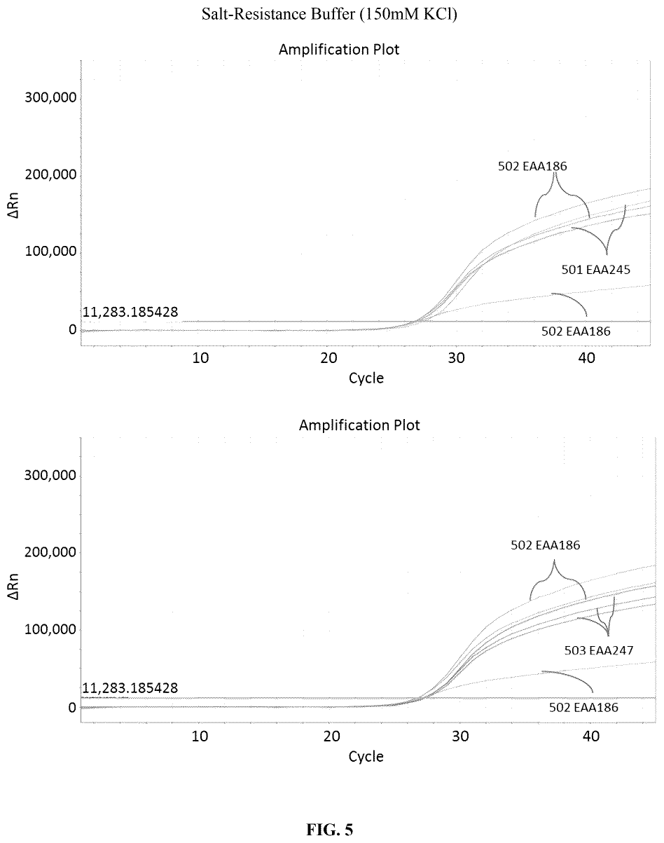

As shown in FIG. 4, mutant Taq DNA polymerases with A516K substitution (EAA186) or with A516R substitution (EAA187) demonstrated enhanced DNA synthesis activity as compared to wild-type Taq DNA polymerase in humic acid PCR reaction.

### EXAMPLE 5

This example illustrates the enhanced DNA synthesis activity of the mutant Taq DNA polymerase with multiple substitutions in PCR reaction with various PCR inhibitors.

The protocols of the salt-resistant PCR reaction, whole blood PCR reaction and humic acid PCR reactions are detailed in EXAMPLEs 1-4 above.

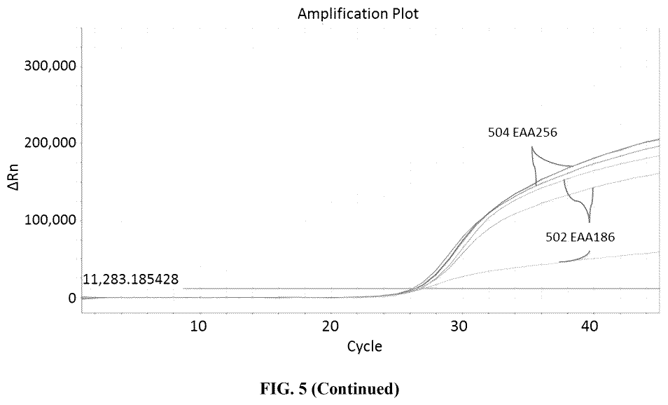

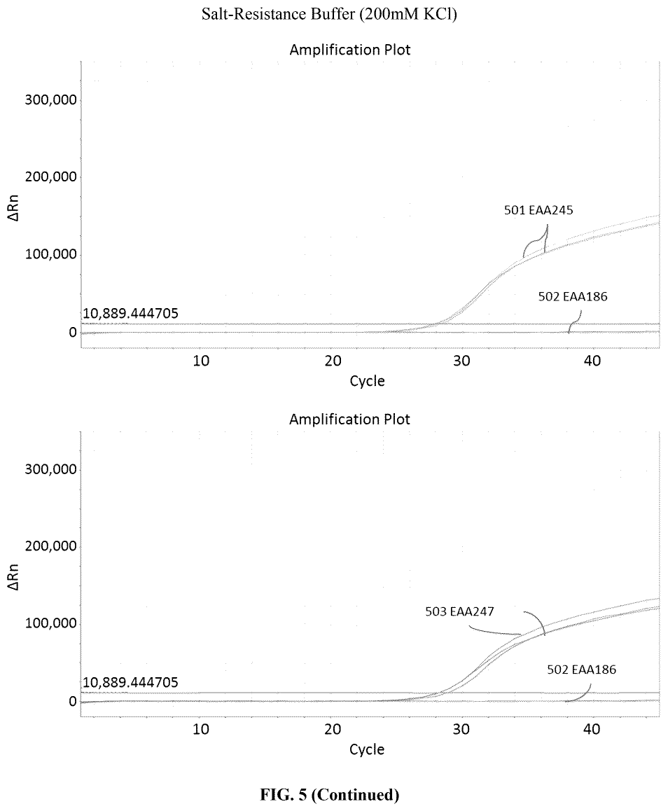

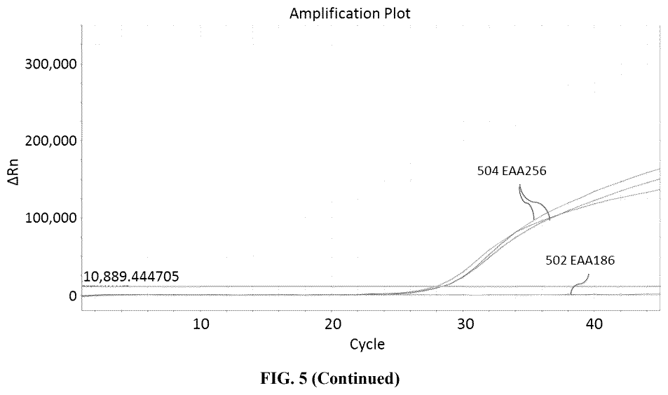

As shown in FIG. 5, mutant Taq with S515K and A516K substitutions (EAA245), mutant Taq with S515R and A516K substitutions (EAA247), and mutant Taq with A516K and S739K substitutions (EAA256) demonstrated further enhanced DNA synthesis activity as compared to mutant Taq with A516K substitution (EAA186) in salt-resistance PCR reactions.

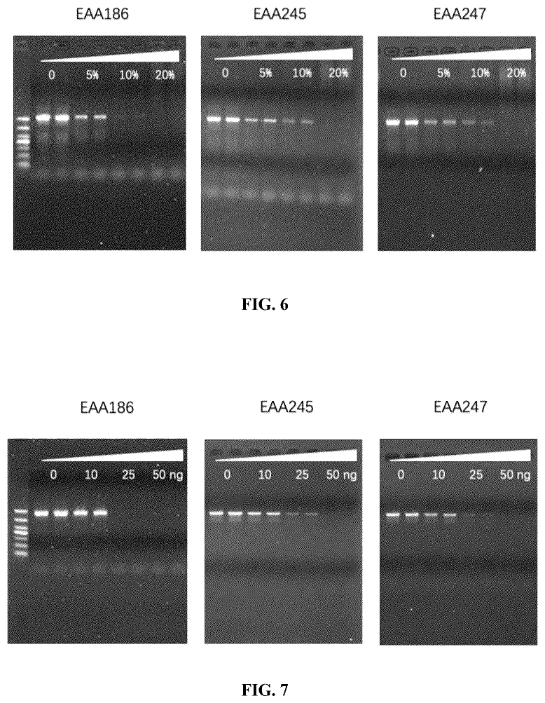

As shown in FIG. 6, mutant Taq with S515K and A516K substitutions (EAA245), and mutant Taq with S515R and A516K substitutions (EAA247) demonstrated further enhanced DNA synthesis activity as compared to mutant Taq with A516K substitution (EAA186) in whole blood PCR reaction.

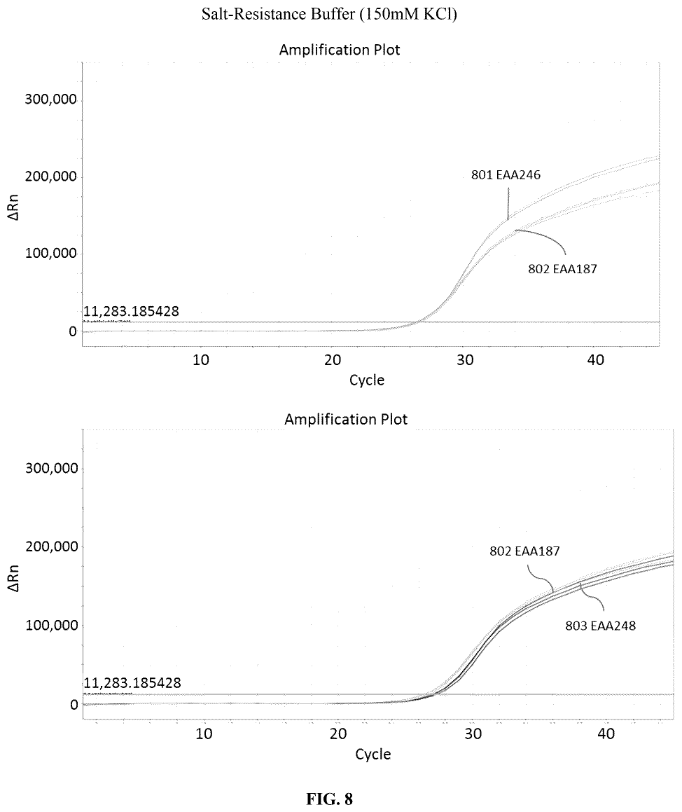

As shown in FIG. 7, mutant Taq with S515K and A516K substitutions (EAA245), and mutant Taq with S515R and A516K substitutions (EAA247) demonstrated further enhanced DNA synthesis activity as compared to mutant Taq with A516K substitution (EAA186) in humic acid PCR reaction.

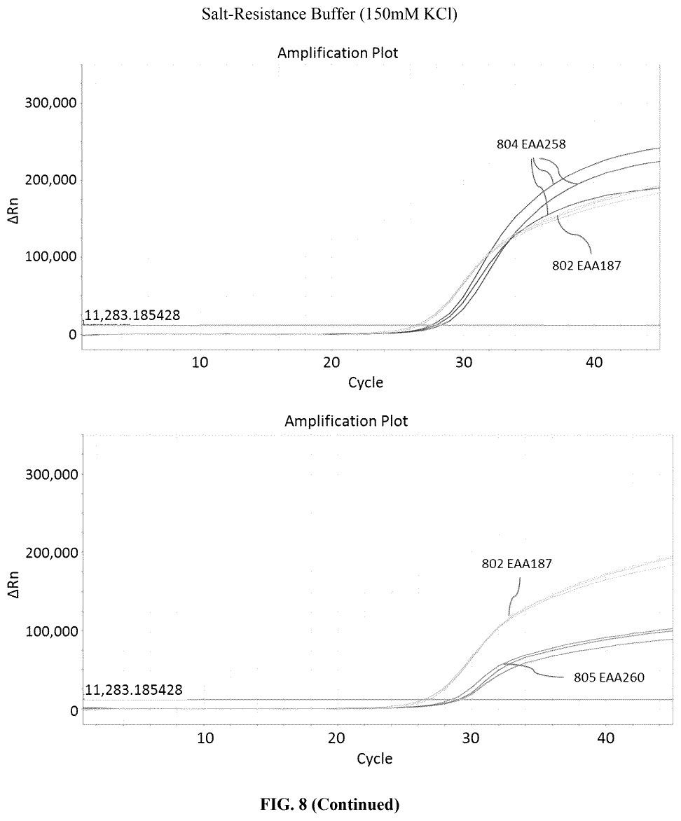

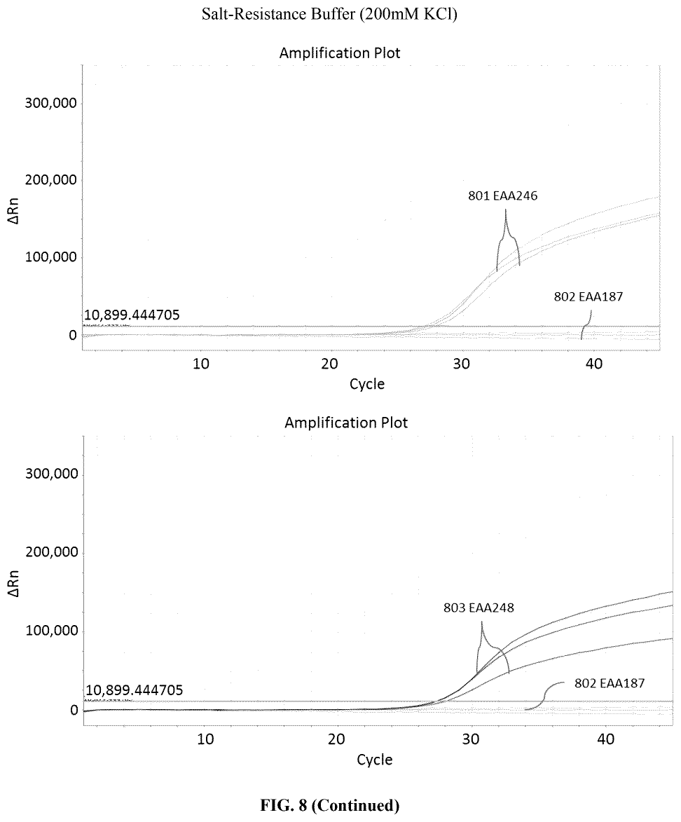

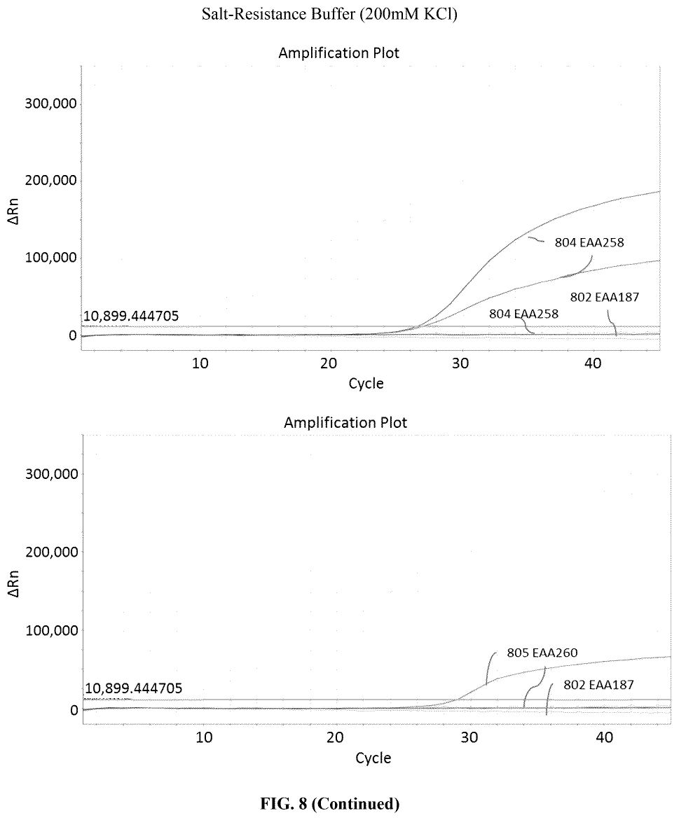

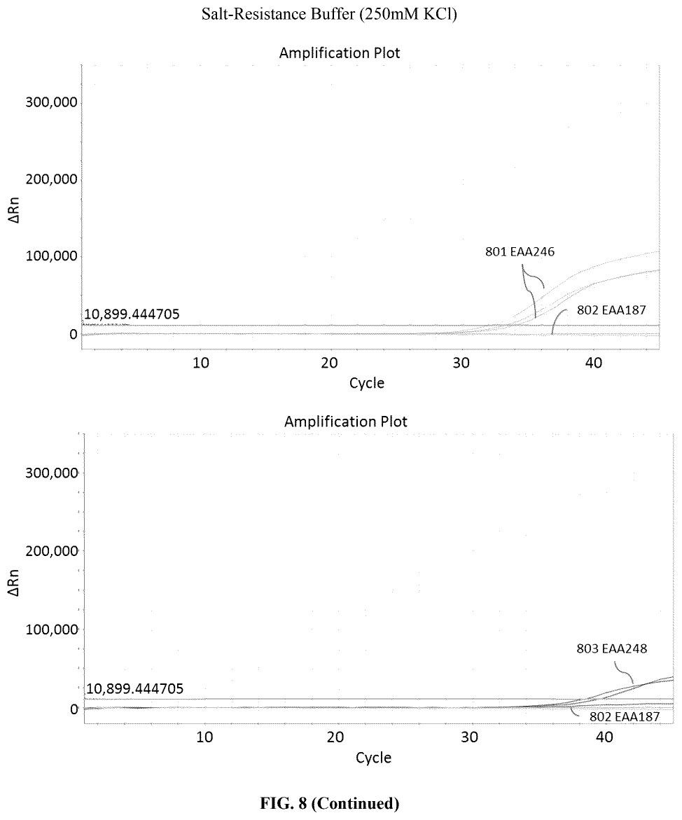

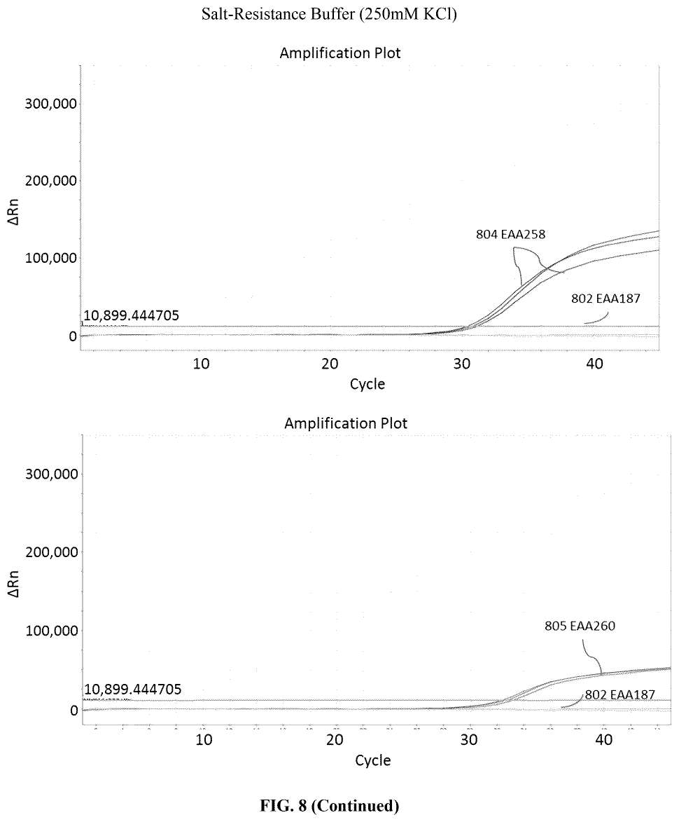

As shown in FIG. 8, mutant Taq with S515K and A516R substitutions (EAA246), mutant Taq with S515R and A516R substitutions (EAA248), mutant Taq with A516R and E742K substitutions (EAA258), mutant Taq with A516R and M747K substitutions (EAA260) demonstrated further enhanced DNA synthesis activity as compared to mutant Taq with A516R substitution (EAA187) in salt-resistance PCR reactions.

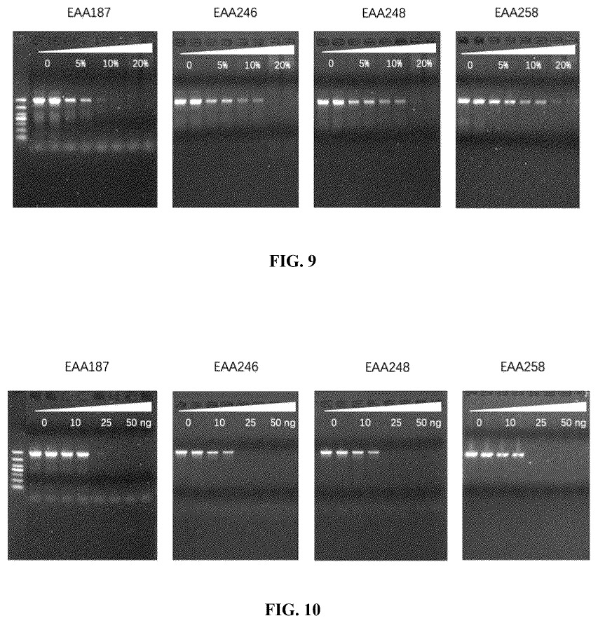

As shown in FIG. 9, mutant Taq with S515K and A516R substitutions (EAA246), mutant Taq with S515R and A516R substitutions (EAA248), and mutant Taq with A516R and E742K substitutions (EAA258) demonstrated further enhanced DNA synthesis activity as compared to mutant Taq with A516R substitution (EAA187) in whole blood PCR reactions.

As shown in FIG. 10, mutant Taq with S515K and A516R substitutions (EAA246), mutant Taq with S515R and A516R substitutions (EAA248), and mutant Taq with A516R and E742K substitutions (EAA258) demonstrated further enhanced DNA synthesis activity as compared to mutant Taq with A516R substitution (EAA187) in humic acid PCR reactions.

### EXAMPLE 6

This example illustrates the enhanced DNA synthesis activity of the mutant Taq DNA polymerase with multiple substitutions in PCR reaction with various PCR inhibitors.

The protocols of the salt-resistant PCR reaction, whole blood PCR reaction and humic acid PCR reactions are detailed in EXAMPLEs 1-4 above.

We generated a series of mutant Taq DNA polymerases with multiple substitutions as shown in the Table below.

| Mutant Taq | Substitutions |
|---|---|
| ETaq-1 | G59W + V155I + L245M + E507K + F749I |
| ETaq-2 | G59W + V155I + L245M + E742K + F749I |
| ETaq-3 | S515R + A516K + S739K |
| ETaq-4 | S515K + A516K + E742K |
| ETaq-5 | S515K + A516R + E742K |
| ETaq-6 | G59W + V155I + L245M + A516R + E742K + F749I |

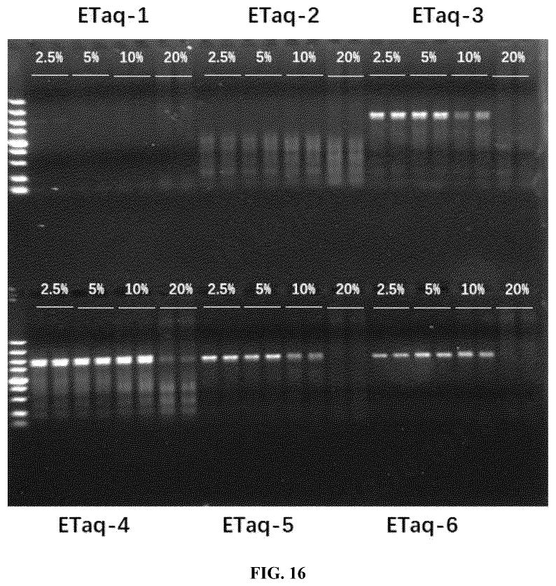

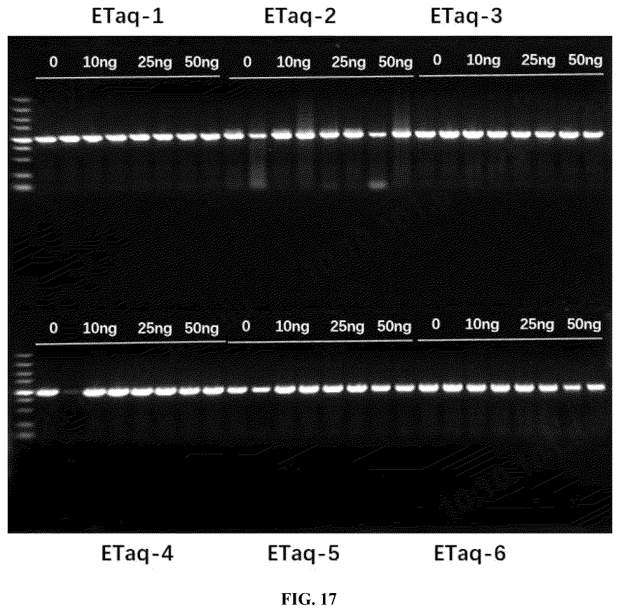

As shown in FIGS. 16 and 17, mutant Taq polymerases containing A516R or A516K substitution demonstrated significantly enhanced DNA synthesis activity in whole blood PCR reactions and humic acid PCR reactions.

What is claimed is:

1. A mutant Taq DNA polymerase comprising at least a substitution of an amino acid residue corresponding to the following residue of a wild-type Taq DNA polymerase:
a serine residue at position 515,
an alanine residue at position 516,
a serine residue at position 739,
an alanine residue at position 743, or
a methionine residue at position 747,
wherein the wild-type Taq DNA polymerase comprises an amino acid sequence corresponding to SEQ ID NO: 1,
wherein the amino acid residue is substituted with an arginine residue, a lysine residue, or a histidine residue.

2. The mutant Taq DNA polymerase according to claim 1, comprising
(1) the amino acid residue corresponding to the serine residue at position 515 of the wild-type Taq DNA polymerase being substituted with an arginine residue or a lysine residue; or
(2) the amino acid residue corresponding to the alanine residue at position 516 of the wild-type Taq DNA polymerase being substituted with an arginine residue or a lysine residue; or
(3) the amino acid residue corresponding to the serine residue at position 739 of the wild-type Taq DNA polymerase being substituted with a lysine residue; or
(4) the amino acid residue corresponding to the alanine residue at position 743 of the wild-type Taq DNA polymerase being substituted with a lysine residue; or
(5) the amino acid residue corresponding to the methionine residue at position 747 of the wild-type Taq DNA polymerase being substituted with a lysine residue.

3. The mutant Taq DNA polymerase according to claim 1, further comprising another one or more substitutions of an amino acid residue, such as the glutamate residue at position 742 of the wild-type Taq DNA polymerase is substituted with a lysine residue.

4. The mutant Taq DNA polymerase according to claim 1, comprising (1) the amino acid residue corresponding to the serine residue at position 515 of the wild-type Taq DNA polymerase being substituted with a lysine residue, and the amino acid residue corresponding to the alanine residue at position 516 of the wild-type Taq DNA polymerase being substituted with a lysine residue; or
(2) the amino acid residue corresponding to the serine residue at position 515 of the wild-type Taq DNA polymerase being substituted with an arginine residue, and the amino acid residue corresponding to the alanine residue at position 516 of the wild-type Taq DNA polymerase being substituted with a lysine residue; or
(3) the amino acid residue corresponding to the alanine residue at position 516 of the wild-type Taq DNA polymerase being substituted with a lysine residue, and the amino acid residue corresponding to the serine residue at position 739 of the wild-type Taq DNA polymerase being substituted with a lysine residue; or
(4) the amino acid residue corresponding to the serine residue at position 515 of the wild-type Taq DNA polymerase being substituted with a lysine residue, and the amino acid residue corresponding to the alanine residue at position 516 of the wild-type Taq DNA polymerase being substituted with an arginine residue; or
(5) the amino acid residue corresponding to the serine residue at position 515 of the wild-type Taq DNA polymerase being substituted with an arginine residue, and the amino acid residue corresponding to the alanine residue at position 516 of the wild-type Taq DNA polymerase being substituted with an arginine residue; or
(6) the amino acid residue corresponding to the alanine residue at position 516 of the wild-type Taq DNA polymerase being substituted with an arginine residue, and the amino acid residue corresponding to the glutamate residue at position 742 of the wild-type Taq DNA polymerase being substituted with a lysine residue; or
(7) the amino acid residue corresponding to the alanine residue at position 516 of the wild-type Taq DNA polymerase being substituted with an arginine residue, and the amino acid residue corresponding to the methionine residue at position 747 of the wild-type Taq DNA polymerase being substituted with a lysine residue.

5. The mutant Taq DNA polymerase according to claim 1, which has an amino acid sequence of at least 80 percent identity to SEQ ID NO: 1.

6. The mutant Taq DNA polymerase according to claim 1, further comprising one or more tags or linkers.

7. The mutant Taq DNA polymerase according to claim 1, wherein the mutant Taq DNA polymerase exhibits a DNA polymerase activity.

8. The mutant Taq DNA polymerase according to claim 1, wherein the mutant Taq DNA polymerase perform better than wild-type Taq DNA polymerase in DNA polymerase activity.

9. The mutant Taq DNA polymerase according to claim 1, wherein the mutant Taq DNA polymerase exhibits a DNA polymerase activity in a PCR reaction containing whole blood, plasma, serum, salt or humic acid.

10. The mutant Taq DNA polymerase according to claim 1, wherein the mutant Taq DNA polymerase exhibits a DNA polymerase activity in a PCR reaction containing (1) at least 5% whole blood, plasma or serum; (2) at least 60 mM salt; or (3) at least 5 ng humic acid.

11. The mutant Taq DNA polymerase according to claim 1, wherein the mutant Taq DNA polymerase exhibits a DNA polymerase activity in a PCR reaction containing (1) any one content in 5%-20% whole blood, plasma or serum; (2) any one content in 60-300 mM salt; or (3) any one content in 5-25 ng humic acid.

12. The mutant Taq DNA polymerase according to claim 1, wherein the mutant Taq DNA polymerase exhibits a DNA polymerase activity in a PCR reaction containing (1) 5%, 10%, or 20% whole blood, plasma or serum; (2) 60 mM, 100 mM, 150 mM, 200 mM, 250 mM or 300 mM salt; or (3) 5 ng, 10 ng, 15 ng, 20 ng, or 25 ng humic acid.

13. The mutant Taq DNA polymerase according to claim 1, wherein the salt is sodium or potassium salt, preferably potassium chloride.

14. A polynucleotide encoding the mutant Taq DNA polymerase of claim 1.

15. A vector comprising the polynucleotide of claim 14.

16. A recombinant host cell suitable for producing a mutant Taq DNA polymerase, comprising the polynucleotide of claim 15.

17. A method of producing a mutant Taq DNA polymerase, comprising the steps of culturing the recombinant host cell of claim 16, thereby giving a culture, and collecting the mutant Taq DNA polymerase from the culture obtained in the above step.

18. A kit for performing a polymerase chain reaction, comprising the mutant Taq DNA polymerase according to claim 1 and a reaction buffer solution.

19. The kit of claim 18, further comprising a primer.

20. A method of performing a polymerase chain reaction, comprising:
incubating the mutant Taq DNA polymerase according to claim 1 with a DNA template and a primer under a condition suitable for the mutant Taq DNA polymerase to perform the polymerase chain reaction, thereby synthesizing a DNA strand complementary to the DNA template.


SEQUENCE LISTING (1 sequence)

SEQ ID NO: 1 - protein, 832 aa, Thermus aquaticus [sequence omitted]